(19) United States
(12) Patent Application Publication
(10) Pub. No.: US 2003/0078595 A1
(43) Pub. Date: Apr. 24, 2003

(54) STEREOTACTIC LOCALIZER SYSTEM WITH DENTAL IMPRESSION

(76) Inventor: ERIC R. COSMAN, BELMONT, MA (US)

Correspondence Address: United States Surgical, a Division of Tyco, Healthcare Group, 150 Glover Avenue, Norwalk, CT, 06856, US

(21) Appl. No.: 08/772,302

(22) Filed: Dec. 23, 1996

Related U.S. Application Data
(63) Continuation of application No. 08/303,859, filed on Sep. 9, 1994, now abandoned.
Application No. 08/303,859 is a continuation-in-part of application No. 08/263,650, filed on Jun. 20, 1994, now abandoned.

(51) Int. Cl. A61B 19/00; A61B 17/50

(52) U.S. Cl. 606/130

(57) ABSTRACT

The present continuation-in-part application shows several specific embodiments of the invention and claims described in the parent patent application. In particular, it shows certain specific geometries of a dental-attached localizer which would be convenient for ear, nose, and throat (ENT) surgical applications or other localization applications related to the front of the face, the skull base, and similar areas. The localizer described in this continuation-in-part has rods and diagonals which allow quasi-coronal CT or other tomographic slices to be made through the face, skull base, and neck anatomy so as to locate structures accurately and repeatedly relative to the coordinate reference frame established by the dental impression piece.

This application is a Continuation-In-Part of application Ser. No. 08/263,650, filed Jun. 20, 1994.

### BACKGROUND TO THE INVENTION AND CONTINUATION-IN-PART

In the parent patent application, a system and method were described involving a dental impression means attached to which a localizer structure is implemented for the purpose of giving reference indicia in tomographic slices through the patient's head and neck region so as to transform or map the image data from said tomographic or 3D volumetric data sets to a coordinate frame associated with said dental impression means. The parent application was general and included applications in neurosurgery, ENT, radiation therapy, and repeated radiographic imaging.

The use in ENT surgery frequently involves taking CT X-ray tomographic slices which are essentially parallel to the plane of face, that is, in a nearly coronal aspect relative to the head. Typically the patient is lying prone; that is, with his stomach downward, on the CT scanning table, and his face looking into the aperture of the scanner. In this configuration, the slices are nearly coronal. It is an objective in ENT surgery to use the tomographic slices to coordinate insertion of tools into critical areas around the nose, sinuses, eyes, and skull base. Great precision is required, since these are dangerous and delicate areas, where 1 or 2 millimeters error in positioning could be harmful or fatal.

Thus, it is an objective of this continuation-in-part to provide embodiments of the claimed invention of the parent application which would be suitable and convenient for the ENT coronal slice application. Furthermore, it is an object of this continuation-in-part, also as claimed in the previous invention, to provide physical index points on the tomographic localizer attached to the dental impression piece which can be used to calibrate, re-calibrate, and locate stereotactic tools, radiation machines, or other instrumentation such as a microscope or ultrasonic head relative to the patient's anatomy at the time of surgery, and all coordinated and cooperatively related to the image slice data and mapping thereof into the frame of reference of the dental impression means.

### DESCRIPTION OF THE FIGURES

FIG. 1 shows a dental-based localizer structure with angled and canted rods and diagonal elements which can enable target localization in axial or coronal slices through the patient's head and face.

FIG. 2 shows another embodiment of a localizer which provides indicia for tomographic slicing in a nearly coronal aspect, as well as index points for referencing a stereotactic instrument during surgery.

### DESCRIPTION OF THE INVENTION

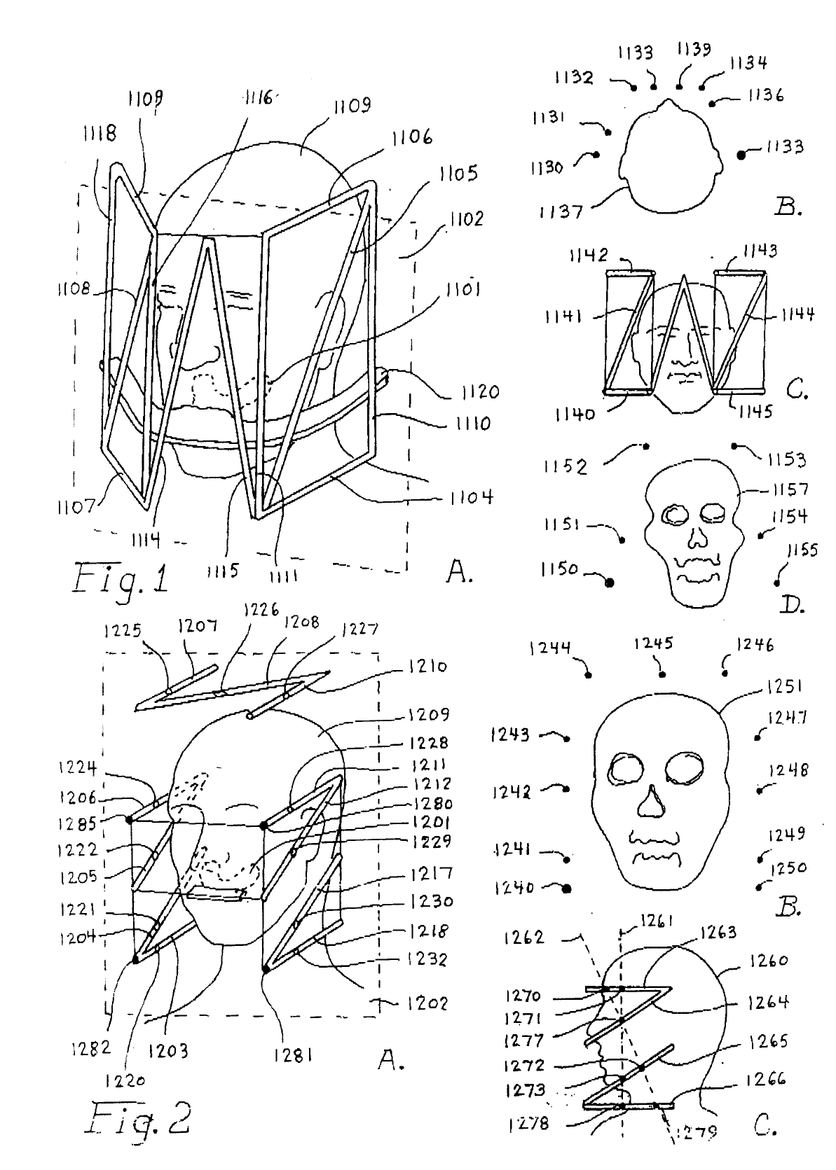

Referring to FIG. 1A, a localizer is shown which is an embodiment of the present invention and the invention described and claimed in the parent application. Included in the invention components is a dental tray 1101, attached to which is a platform 1120. The platform supports vertical, horizontal, and diagonal elements. The vertical elements 1110, 1111, 1116, 1118, together with the diagonal elements 1105, 1115, 1114, and 1108, give rise to varying index points in tomographic scans so as to determine a mapping from a two-dimensional slice image in an axial plane or quasi-axial plane to a three-dimensional coordinate system associated with the dental tray 1101, which is directly molded to the teeth and thus directly affixed to the patient's skull via the patient's upper and/or lower teeth. Also shown in FIG. 1A are horizontal rods 1104, 1106, 1107, and 1109, which enable a quasi-coronal plane such as plane 1102 to include index data that in turn enables a mapping from a quasi-coronal slice through the patient's face and related anatomy so as to map the data from this CT image slice to the coordinate reference frame associated with the dental tray 1101.

FIG. 1B shows the index data as it might appear from a quasi-axial CT slice through the anatomy and the localizer of FIG. 1A. The index points from the vertical rods are shown by spots 1133, 1134, 1132, and 1130, and the index marks from the diagonal rods are shown as 1136, 1139, 1138, and 1131. The patient's anatomy is also shown by the outline 1137. The position of the index points and their relative location enable such a two-dimensional planar data set as FIG. 1B to be mapped into the three-dimensional coordinate system relative to the dental tray 1101 and the attached platform 1120 according to the teachings and invention of Russell Brown, U.S. Pat. No. 4,608,977. Such a mapping is alluded to in the invention of the parent patent application with respect to this type of implementation. Furthermore, shown in FIG. 1C is a front-on view of the localizer of FIG. 1A, and here is shown the horizontal rods 1140, 1142, 1143, and 1145, as well as the diagonals 1141 and 1144 which enabled a quasi-coronal slice such as 1102 to include sufficient index data to map from such a quasi-coronal slice, again to the dental and platform coordinate system of elements 1101 and 1120. For example, in FIG. 1B there is shown a slice through such a structure which is not perfectly parallel to the vertical rods of the localizer, and there are the associated index points 1150, 1152, 1153, and 1155 associated with the horizontal rods and the index points 1151 and 1154 associated with the diagonal elements. The fact that the lines associated with the point pairs 1150, 1152 and 1155, 1153 are not parallel indicates that the plane of the slice is not parallel to the vertical rods. The location of all of these index points and the relationship of the angle included between the line elements 1150 to 1152, compared to the line element 1153 to 1154, give further indication about the inclination of the plane relative to the localizer, and thus the mapping alluded to above can be done. Thus, each point of the anatomy, illustrated by 1157, can be so mapped into the frame of reference of the dental tray.

Referring to FIG. 2, another embodiment of the invention of the parent patent application is shown with a localizer, including a dental try 1201 attached cooperatively to rod and diagonal structures. In this case, the horizontal rods are illustrated by 1203, 1224, 1207, 1124, 1125, 1210, 1211, and 1218, and the diagonal rod elements are illustrated by elements 1204, 1205, 1208, 1212, and 1217. A mathematical plane, illustrated by the dashed plane 1202, is shown in a nearly coronal aspect to the patient's face, and it intersects these various structures at the points 1220, 1221, 1222, 1224, 1225, 1226, 1227, 1228, 1229, 1230, 1232. FIG. 2B illustrates how these index points would look in a typical two-dimensional X-ray CT slice, such as plane 1202. Index marks for the horizontal rods are 1240, 1243, 1244, 1246, 1247, 1250, and the index points for the diagonal elements are illustrated as 1241, 1242, 1245, 1248, and 1249. The patient's anatomy is illustrated by the diagram 1251. By determining the two-dimensional coordinate positions of each of these index points, as well as any points in the anatomy in a two-dimensional slice, a transformation, calculation, or mapping can be made by computer, manually, or graphically between this data set and the three-dimensional coordinate reference frame of the dental tray and its associated attachment structure to the diagonal and horizontal rods. The pattern of the index points from the diagonal and parallel rods shown in FIG. 2B make determinable not only the coordinate point mapping but also the inclination of the plane of the slice. FIG. 2 shows a lateral elevation view of the patient's head with the localizer structure in place. Horizontal rods are illustrated by 1263 and 1266 on one side of the head, The diagonal rods 1264 and 1265 are also shown for the structure on one side of the face and head. The projected plane of two CT slices is shown by 1262 and 1261. The intersection points for each of these planes are illustrated as 1271, 1277, 1273, and 1278 for plane 1261 and as 1270, 1277, 1272, and 1279 for the plane 1262. The relationship of these points can be seen to change, depending on the angle of the plane and the position of the plane, and it is this kind of varying relationship of the index points, one relative to the other, which enables the full plane inclination angles as well as mapping of points within the plane to be done according to the patent of Brown. Also, the use of the horizontal rods in this particular example and in the example of FIG. 1 in conjugation with the diagonal rods make it possible to do the mapping easily for an arbitrary CT slice plane. Not shown in FIG. 2C are the index points for the upper elements 1207, 1208, and 1202 with their associated image fiducial points 1244, 1245, and 1246. These may also be used for an appropriate transformation of coordinates. With the use of two diagonals as shown on each of the side structures of FIG. 2A, the top upper N-shaped structure gives added information which is duplicated but may improve accuracy. The upper N-shaped structure is not essential for these quasi-coronal slices, but is shown here for completeness, as it may be desired to have only one diagonal element on each of the structures to the left and right of the patient's head. It should also be pointed out that not all of these rods and diagonal elements are necessary to do the mapping or transformation; nor is it necessary to do a full computer code matrix transformation to perform the mapping. A simple triangular plate structure, single rod and single diagonal structure, varying numbers of rod and diagonal structures on the left, right, top, and other portions of the head, curvilinear rod or diagonal elements, spiral structures, arrays of index points, arrays of rods and pins of varying lengths and configurations could all be used to implement the basic invention of this continuation-in-part and of its parent patent application. They are all embodiments of graphic reference means which may be attached to a dental reference frame that can be seen in tomographic or three-dimensional imaging processes that enable the frame of reference of the data from the image processing means to be transformed or mapped into the coordinate reference frame associated with the patient's anatomy, in this case the skull, which is attached to the dental tray.

The devices such as those illustrated in FIG. 1 and FIG. 2 are particularly convenient in the ENT field, where quasi-coronal slices through the patient's head are used. They provide an accurate and detailed mapping from the slice data to the patient's anatomy which can then be used in turn to provide accurate approach, angulation, and calculation to targets and paths through the patient's nose, mouth, or other facial aspects into interior anatomical target points for surgery. Use for maxofacial, nasal, pituitary, acoustic, head, throat, and larynx surgery is an obvious application, as discussed in the parent application. The extrapolation to stereotactic target coordination for radiation therapy is also a consequence and application, as described in the parent application. Furthermore, in the diagram of FIG. 2A there are reference points or positions illustrated by 1282, 1285, 1280, and 1281, which are identifiable physical points of known or determinable coordinates associated with the localizer structure that can be accessed during the imaging and also potentially during the surgical, set-up, or calibration phase of the treatment or surgery. As illustrated in the parent invention, these points can be touched off by or located from, for example, a stereotactic digitizer or navigator which may be mechanical, optical, ultrasonic, radiofrequency, magnetic, or otherwise coupled to an interface to a computer graphic workstation, thereby enabling that navigator to be calibrated relative to the patient's anatomy and recalibrated, if necessary, by simple insertion of the dental tray into the patient's mouth.

Included within the scope of this continuation-in-part and in the parent application is use of such identifiable physical points associated with the localizer and/or the dental applicator. Those skilled in the art can make further variations of this present disclosed embodiments for the use in ENT surgery and surgery and therapy in and around the face. It should be noted that although the embodiments in FIG. 1 and FIG. 2 show what is called horizontal rods and diagonal rods thereto, it is possible to make such a localizer to function as described by having only what one would say is diagonal rods; that is, rods that are angled one relative to the other, and not necessarily sets of parallel rods at all. For example, systems of V-shaped rods could be used or merely a single V-shaped structure could be used to give an index of the position of the tomographic slice. The combination of V-shaped or N-shaped structures, or other structures thereof, could be used in conjugation with additional knowledge about the orientation of the CT image plane such as its degree of parallelity to external apparatus to effect such a data mapping. Combinations of index points and rods could be used to effect the purpose which is claimed in this continuation-in-part and the parent invention.

Having described the continuation-in-part, I therefore claim as letters patent to this invention the following:

1. A dental-based localize that can be used for locating targets in the anatomy of a patient in the region of the ear, nose, throat, and skull base, including:
(a) a dental piece that can be fit to the teeth of said patient so as to locate thereby said dental piece said anatomy of said patient;
(b) graphic reference means that is adapted to be attached to and located in position relative to said dental piece, said graphic reference means having at least one diagonal element which will produce varying index data in a coronal tomographic slice from a computer tomographic imaging machine, said coronal tomographic slice being oriented substantially in an arbitrary plane that is non-parallel to the plane of the teeth and/or substantially coronal to the head of said patient, said coronal tomographic slice intersecting said graphic reference means and said anatomy of said patient, whereby said index data from arbitrary said coronal tomographic slice can be used to reference the position of said coronal tomographic slice relative to said dental piece and therefore to said anatomy of said patient.

2. The apparatus of claim 1 wherein said graphic reference means includes a multiplicity of said diagonal elements which are oriented at angles with respect to each other such that said index data includes patterns of fiducial points such that the distances between said fiducial points give position data that can be used to reference the position of said coronal tomographic slice relative to said dental piece, and therefore to said anatomy.

3. The apparatus of claim 1 wherein said graphic reference means includes parallel rod means, and said diagonal elements include at least one diagonal rod which is non-parallel to said parallel rod means such that when said coronal tomographic slice intersects said parallel rod means and said diagonal rod, said computer tomographic slice produces fiducial marks in said index data which can be used to reference said coronal tomographic slice with respect to said dental piece, and therefore with respect to the anatomy, which is included in said coronal tomographic slice, said anatomy including that near the face of said patient.

4. The apparatus claim 3 there sufficient number of diagonal rods and parallel rod means that there is a sufficient number of said fiducial marks in said index data for an arbitrary coronal tomographic slice through said graphic reference means and said anatomy that a mapping and/or transformation can be performed using said index data to relate the position of points of said anatomy seen in said tomographic slice to the frame of reference of said dental tray and of said graphic reference means.

5. The apparatus of claim 1 and further including physical locating points which are adapted to be coupled to and located at predetermined positions with respect to said dental piece and which are adapted to be located in space by the pointing element of a stereotactic instrument, said pointing element of said stereotactic instrument being cooperatively coupled to said stereotactic instrument so that the orientation and/or location of said pointing element relative to said stereotactic instrument is readable as stereotactic instrument data, whereby said stereotactic instrument data associated with said physical locating points is used to relate the position of said stereotactic instrument to said dental piece, and thereby to relate said stereotactic instrument to said anatomy of said patient as determined by said coronal tomographic slice.
